(19) United States
(12) Patent Application Publication
(10) Pub. No.: US 2014/0037749 A1
(43) Pub. Date: Feb. 6, 2014

(54) Delivery Scaffolds and Related Methods of Use

(71) Applicant: NORTHWESTERN UNIVERSITY, (US)

(72) Inventors: Lonnie D. Shea, Chicago, IL (US); William L. Lowe, Winnetka, IL (US); Christopher B. Rives, Natick, MA (US)

(73) Assignee: NORTHWESTERN UNIVERSITY, Evanston, IL (US)

(21) Appl. No.: 13/828,293

(22) Filed: Mar. 14, 2013

Related U.S. Application Data
(62) Division of application No. 12/359,873, filed on Jan. 26, 2009.
(60) Provisional application No. 61/023,358, filed on Jan. 24, 2008.

(51) Int. Cl. A61L 27/00; A61L 27/26; A61L 27/22; A61L 27/20; A61L 27/18; A61L 27/36

(52) U.S. Cl. CPC A61L 27/00; A61L 27/18; A61L 27/36; A61L 27/227; A61L 27/3604; A61L 27/20; A61L 27/26
USPC 424/501; 424/400; 424/489; 424/93.1; 514/1.1; 424/93.7; 514/44 R

(57) ABSTRACT

The present invention relates to delivery systems. In particular, the present invention provides microporous scaffolds having thereon agents (e.g., extracellular matrix proteins, exendin-4) and biological material (e.g., pancreatic islet cells). In some embodiments, the scaffolds are used for transplanting biological material into a subject. In some embodiments, the scaffolds are used in the treatment of diseases (e.g., type 1 diabetes), and related methods (e.g., diagnostic methods, research methods, drug screening).

This application claims priority to U.S. Provisional Patent Application Ser. No. 61/023,358, filed Jan. 24, 2008, the disclosure of which is herein incorporated by reference in its entirety.

### STATEMENT REGARDING FEDERALLY SPONSORED RESEARCH OR DEVELOPMENT

This invention was made with government support under F31 EB007118, R21 DK067833, and R01 EB003805 awarded by the National Institutes of Health. The government has certain rights in the invention.

### FIELD OF THE INVENTION

The present invention relates to delivery systems. In particular, the present invention provides microporous scaffolds having thereon agents (e.g., extracellular matrix proteins, exendin-4) and biological material (e.g., pancreatic islet cells). In some embodiments, the scaffolds are used for transplanting biological material into a subject. In some embodiments, the scaffolds are used in the treatment of diseases (e.g., type 1 diabetes), and related methods (e.g., diagnostic methods, research methods, drug screening).

### BACKGROUND OF THE INVENTION

Islet transplantation is the transplantation of isolated islets from a donor pancreas and into another person. It is an experimental treatment for type 1 diabetes mellitus. Once transplanted, the islets begin to produce insulin, actively regulating the level of glucose in the blood. Islets are usually infused into the patient's liver (Lakey J, Burridge P, Shapiro A (2003). “Technical aspects of islet preparation and transplantation”. Transpl Int 16 (9): 613-632). The patient's body, however, will treat the infused islets just as it would any other introduction of foreign tissue: the immune system will attack the islets as it would a viral infection, leading to the risk of transplant rejection. Thus, the patient needs to undergo treatment involving immunosuppressants, which reduce immune system activity.

Although beta-cell replacement via transplantation of allogeneic islets has been explored as a potential curative treatment for type 1 diabetes, clinical islet transplantation has thus far yielded disappointing results, with less than 10% of those transplanted remaining insulin independent after five years (see, e.g., Ryan E A, Paty B W, Senior P A, et al. Five-year follow-up after clinical islet transplantation. Diabetes 2005; 54 (7): 2060).

Improved methods for islet transplantation are needed.

### SUMMARY OF THE INVENTION

The present invention relates to delivery systems. In particular, the present invention provides microporous scaffolds having thereon agents (e.g., extracellular matrix proteins, exendin-4) and biological material (e.g., pancreatic islet cells). In some embodiments, the scaffolds are used for transplanting biological material into a subject. In some embodiments, the scaffolds are used in the treatment of diseases (e.g., type 1 diabetes), and related methods (e.g., diagnostic methods, research methods, drug screening).

In experiments conducted during the course of development of embodiments for the present invention, a scaffold design comprising a thin, non-porous center layer sandwiched between two highly porous outer layers is provided that exhibits an enhanced capacity for delivery of, for example, pharmaceutical agents, DNA, RNA, and/or biological material (e.g., pancreatic islet cells). In experiments conducted during the course of development of embodiments for the present invention, the layered scaffold design was shown to achieve sustained delivery of exendin-4 for 2 months, and demonstrated increased blood glucose control in diabetic mice that were transplanted with pancreatic islets on exendin-4 releasing scaffolds relative to controls.

In some embodiments, the scaffold comprises three layers, an inner layer and two outer layers, where the inner layer is less porous than the outer layers. In some embodiments, the inner layer is substantially non-porous or is non-porous. In some embodiments, a chemical or biological agent is associated with the inner layer. In some embodiments, the chemical or biological agent is encapsulated in particles (e.g., microspheres, such as poly(lactide-co-glycolide) (PLG) microspheres). The present invention is not limited by the nature of the chemical or biological agents. Such agents include, but are not limited to, proteins, nucleic acid molecules, small molecule drugs, lipids, carbohydrates, cells, cell components, and the like. In some embodiments, two or more (e.g., 3, 4, 5, . . . ) different chemical or biological agents are included in the inner layer. In some embodiments, the different agents are configured (e.g., in the appropriate particles) for different release rates. For example, a first agent may release over a period of 30 days while a second agent releases over a longer period of time (e.g., 60 days, 70 days, 90 days, etc.). In some embodiments, the inner layer is substantially free of salt or is free of salt. In some embodiments, the inner layer is configured for slow-release of the biological or chemical agents. In some embodiments, the slow release provides release of biologically active amounts of the agent over a period of at least 30 days (e.g., 40 days, 50 days, 60 days, 70 days, 80 days, 90 days, 100 days, 180 days, etc.). In some embodiments, the outer layers are configured to be sufficiently porous to permit ingrowth of cells into the pores. The size of the pores may be selected for particular cell types of interest and/or for the amount of ingrowth desired.

In experiments conducted during the course of development of embodiments for the present invention, extracellular matrix proteins adsorbed to microporous scaffolds enhance the function of transplanted islets, with collagen IV and/or exendin-4 maximizing graft function relative to the other proteins tested.

Accordingly, in certain embodiments, the present invention provides microporous scaffolds having thereon cells or other biological or chemical agents. Where cells are employed, the scaffolds are not limited to a particular type of cells. In some embodiments, the scaffolds have thereon pancreatic islet cells. In some embodiments, the microporous scaffolds additionally have thereon ECM proteins and/or exendin-4. The scaffolds are not limited to a particular type of microporous scaffold. In some embodiments, the scaffold has a thin nonporous layer positioned between two highly porous outer layers. In some embodiments, the nonporous layer has thereon pharmaceutical agents, DNA, RNA, extracellular matrix proteins, exendin-4, etc. In certain embodiments, the present invention provides methods for transplanting pancreatic islet cells with such scaffolds. In certain embodiments, the present invention provides methods for treating type 1 diabetes (e.g., increasing blood glucose control; restoring euglycemia) in a subject with such scaffolds.

### BRIEF DESCRIPTION OF THE DRAWINGS

FIG. 1 shows a diagram of layered scaffold design. A non-porous center layer is sandwiched between two identical, highly porous outer layers. The center layer can be designed to function as an effective drug delivery device, while the outer layers provide an optimal physical structure that allows for cell seeding tissue infiltration. The non-porous center layer can minimize drug loss during the particulate leaching step, and can slow the drug release. A premise of this design is that it allows the properties of the different layers to be optimized independently from each other, which is advantageous when constructing a scaffold that serves two different purposes (e.g., a physical structure and a drug delivery device).

FIG. 2 shows in vitro release of exendin-4 from layered scaffolds. The left panel shows exendin-4 release kinetics for the outer scaffold layers, and the right panel shows release kinetics for the center scaffold layer. The outer layers provide a burst release profile, with most of the protein being released over a period of 2 days. The center layer provides a sustained release of exendin-4 over a period of 2 months.

FIG. 3 shows blood glucose levels for diabetic mice transplanted with 75 islets on control or exendin-4 loaded scaffolds. Islets transplanted on exendin-4 releasing scaffolds showed improved glucose control relative to islets transplanted on control scaffolds.

FIG. 4 shows characterization of DNA incorporation and release for layered scaffolds. DNA (800 μg) was loaded into the center scaffold layer, which consisted of either 2 mg or 3 mg of polymer. The layered scaffold design allows for high DNA incorporation efficiencies (>70%), as seen in the right panel. The left panel shows DNA release kinetics for scaffolds with a center layer composed of 2 mg of polymer. The DNA was released rapidly in vitro over a period of 3 days. The bottom panel is an image of an agarose gel showing the conformation of DNA released from scaffolds as a function of time. Lane 1: DNA ladder, lane 2: initial DNA, lanes 3-8: 8 hrs, 24 hrs, 3 days, 7 days, 14 days, and 21 days. A large proportion of the released DNA remained in the supercoiled conformation for all time-points, although there was a gradual loss of the supercoiled conformation and an increase in the appearance of nicked and linear conformation as time progresses.

FIG. 5 shows in vivo luciferase transgene expression following implantation of layered DNA scaffolds into the epididymal fat of C57BIJ6 mice. Luciferase expression was detected through 2 weeks following scaffold implantation and both DNA doses tested (800 μg and 400 μg) were found to provide similar levels of gene expression at all time-points measured.

FIG. 6 shows protein adsorption to scaffolds. Photomicrographs of scaffolds stained with picrosirius red after 1 mg/ml collagen IV was adsorbed. The scaffolds were treated by base hydrolysis (A) or were untreated (B). Negative control for base-hydrolyzed scaffold by incubation with PBS (C). Indicator marks at bottom of images are 1 mm apart.

FIG. 7 shows glucose regulation following islet transplantation. (A) Blood glucose levels from day 0 thru day 300 post-transplantation for mice implanted with scaffolds coated with collagen IV (filled circle, solid line), fibronectin (filled rectangle, dashed line), laminin (open circle, dashed line) and serum proteins (filled triangle, dashed line), or control scaffolds without islets (open rectangle, solid line). Values represent the mean glucose level at each time point (n=7 for collagen IV group, n=8 for all other groups). Error bars omitted for clarity. (B) The fraction of diabetic animals that converted to euglycemia over time for scaffolds coated with collagen IV (solid line), fibronectin (dashed line), laminin (dash-dot line), and serum proteins (dot-dot line). The symbol *** represent statistical significance at P<0.001 for collagen IV relative to all other conditions.

FIG. 8 shows changes in body weight following islet transplantation. Percent change in body weight from day 0 (day of transplant) is plotted as a function of time for scaffolds coated with collagen IV (filled circle, solid line), fibronectin (filled rectangle, dashed line), laminin (open circle, dashed line), and serum proteins (filled triangle, dashed line).

FIG. 9 shows intraperitoneal glucose tolerance tests. An IPGTT was performed at four weeks (A,B) and forty weeks (C,D) following islet transplantation. (A,C) Blood glucose levels as a function of time following glucose challenge for scaffolds. (B,D) Areas under the glucose challenge curves were calculated. Reported values represent the mean glucose levels at each time point±SEM (at four weeks: n=7 for the collagen IV group, n=5 for the fibronectin group, n=6 for the laminin group, n=4 for the serum group, and n=3 for the normal control group; at forty weeks: n=7 for the collagen IV group, n=8 for the fibronectin group, n=8 for the laminin group, n=6 for the serum group, and n=3 for the normal control group). *P<0.05 compared to the fibronectin group, ⁺P<0.05 compared to the laminin group, ⁻P<0.05 compared to the serum group, **P<0.01, ***P<0.001.

### DETAILED DESCRIPTION

Type 1 diabetes mellitus (T1DM) affects an estimated 1.5 million Americans (see, e.g., Eiselein L, Schwartz H J, Rutledge J C. The challenge of type 1 diabetes mellitus. Ilar J 2004; 45 (3): 231) and is characterized by autoimmune-mediated destruction of pancreatic beta-cells, which results in absolute insulin deficiency (see, e.g., Eisenbarth G S. Type I diabetes mellitus. A chronic autoimmune disease. N Engl J Med 1986; 314 (21): 1360; Hamalainen A M, Knip M. Autoimmunity and familial risk of type 1 diabetes. Curr Diab Rep 2002; 2 (4): 347; Yoon J W, Jun H S. Autoimmune destruction of pancreatic Beta cells. Am J Ther 2005; 12 (6): 580; Wilson D B. Immunology: Insulin auto-antigenicity in type 1 diabetes. Nature 2005; 438 (7067): E5). While careful glucose monitoring combined with exogenous insulin administration can effectively control acute glycemia, secondary microvascular and macrovascular complications eventually afflict most type 1 diabetic subjects (see, e.g., Mohsin F, Craig M E, Cusumano J, et al. Discordant trends in microvascular complications in adolescents with type 1 diabetes from 1990 to 2002. Diabetes Care 2005; 28 (8): 1974; Nathan D M. Management of insulin-dependent diabetes mellitus. Drugs 1992; 44 Suppl 3: 39; Nathan D M. Long-term complications of diabetes mellitus. N Engl J Med 1993; 328 (23): 1676). Although beta-cell replacement via transplantation of allogeneic islets has been explored as a potential curative treatment, clinical islet transplantation has thus far yielded disappointing results, with less than 10% of those transplanted remaining insulin independent after five years (see, e.g., Ryan E A, Paty B W, Senior P A, et al. Five-year follow-up after clinical islet transplantation. Diabetes 2005; 54 (7): 2060). Moreover, the stringent inclusion criteria for and shortage of donors, coupled with the requirement for two to four donor pancreata per recipient, limit the potential of this approach (see, e.g., Balamurugan A N, Bottino R, Giannoukakis N, Smetanka C. Prospective and challenges of islet transplantation for the therapy of autoimmune diabetes. Pancreas 2006; 32 (3): 231; Hering B J. Achieving and maintaining insulin independence in human islet transplant recipients. Transplantation 2005; 79 (10): 1296; Hering B J, Kandaswamy R, Ansite J D, et al. Single-donor, marginal-dose islet transplantation in patients with type 1 diabetes. Jama 2005; 293 (7): 830).

Reasons for the limited success of islet transplantation are multi-factorial and related to the loss of vascular connections (see, e.g., Lai Y, Schneider D, Kidszun A, et al. Vascular endothelial growth factor increases functional beta-cell mass by improvement of angiogenesis of isolated human and murine pancreatic islets. Transplantation 2005; 79 (11): 1530; Pileggi A, Molano R D, Ricordi C, et al. Reversal of Diabetes by Pancreatic Islet Transplantation into a Subcutaneous, Neovascularized Device. Transplantation 2006; 81 (9): 1318) and disruption of cell-matrix contacts that occur during the isolation procedure (see, e.g., Balamurugan A N, Bottino R, Giannoukakis N, Smetanka C. Prospective and challenges of islet transplantation for the therapy of autoimmune diabetes. Pancreas 2006; 32 (3): 231). Basement membrane proteins present between intraislet endothelial and endocrine islet cells are primarily collagen IV, laminin and fibronectin. These proteins engage integrins on the surface of islet cells to mediate adhesion, provide structural support and activate intracellular chemical signaling pathways (see, e.g., Hamamoto Y, Fujimoto S, Inada A, et al. Beneficial effect of pretreatment of islets with fibronectin on glucose tolerance after islet transplantation. Horm Metab Res 2003; 35 (8): 460; Jiang F X, Naselli G, Harrison L C. Distinct distribution of laminin and its integrin receptors in the pancreas. J Histochem Cytochem 2002; 50 (12): 1625; Kaido T, Yebra M, Cirulli V, Montgomery A M. Regulation of human beta-cell adhesion, motility, and insulin secretion by collagen IV and its receptor alpha 1beta1. J Biol Chem 2004; 279 (51): 53762). During enzymatic digestion of the exocrine pancreas, these extracellular matrix (ECM) proteins are degraded, which interrupts cell-matrix interactions (see, e.g., Paraskevas S, Maysinger D, Wang R, Duguid T P, Rosenberg L. Cell loss in isolated human islets occurs by apoptosis. Pancreas 2000; 20 (3): 270; Thomas F, Wu J, Contreras J L, et al. A tripartite anoikis-like mechanism causes early isolated islet apoptosis. Surgery 2001; 130 (2): 333; Thomas F T, Contreras J L, Bilbao G, Ricordi C, Curiel D, Thomas J M. Anoikis, extracellular matrix, and apoptosis factors in isolated cell transplantation. Surgery 1999; 126 (2): 299). Early islet cell death following transplantation may be related, for example, to a lack of integrin signaling resulting in apoptosis (see, e.g., Thomas F T, Contreras J L, Bilbao G, Ricordi C, Curiel D, Thomas J M. Anoikis, extracellular matrix, and apoptosis factors in isolated cell transplantation. Surgery 1999; 126 (2): 299). Islets cultured on matrices containing ECM components, on the other hand, exhibited improved survival in vitro (see, e.g., Lucas-Clerc C, Massart C, Campion J P, Launois B, Nicol M. Long-term culture of human pancreatic islets in an extracellular matrix: morphological and metabolic effects. Mol Cell Endocrinol 1993; 94 (1): 9). Accordingly, the provision of a matrix to support islet attachment is an important requirement for maintaining the function and viability of transplanted islets.

Microporous, biocompatible, biodegradable scaffolds fabricated from poly(lactide-co-glycolide) (PLG) have been successfully used as platforms for islet transplantation in mice (see, e.g., Blomeier H, Zhang X, Rives C, et al. Polymer scaffolds as synthetic microenvironments for extrahepatic islet transplantation. Transplantation 2006; 82 (4): 452). This type of scaffold offers distinct advantages, including, for example, (i) a high surface area/volume ratio to enable nutrient and waste transport, (ii) an interconnected internal pore structure to allow for cell and blood vessel infiltration, (iii) sufficient mechanical rigidity to provide a platform for cell attachment and ease of implantation, and (iv) the ability to degrade over time, allowing for complete integration into the surrounding tissue. In addition to providing structural support, the scaffold surface can be modified with non-diffusible molecules, such as, for example, ECM components, to mediate cellular interactions that are necessary for cell attachment, growth and proliferation (see, e.g., Lutolf M P, Hubbell J A. Synthetic biomaterials as instructive extracellular microenvironments for morphogenesis in tissue engineering. Nat Biotechnol 2005; 23 (1): 47). This surface modification allows manipulation of the local microenvironment so that the impact of factors in isolation or combination on graft efficacy can be determined.

In experiments conducted during the course of development of embodiments for the present invention, the ability and specificity of ECM proteins to promote the long-term function of islets that were transplanted onto microporous scaffolds coated with collagen IV, laminin or fibronectin, and implanted into a mouse model of diabetes was investigated. The epididymal fat pad was selected as the site of implantation due to its surgical accessibility, vascularization, and structural similarity to the greater omentum in humans (a potential extrahepatic site for clinical islet transplantation) (see, e.g., Blomeier H, Zhang X, Rives C, et al. Polymer scaffolds as synthetic microenvironments for extrahepatic islet transplantation. Transplantation 2006; 82 (4): 452; Chen X, Zhang X, Larson C, Chen F, Kissler H, Kaufman D B. The epididymal fat pad as a transplant site for minimal islet mass. Transplantation 2007; 84 (1): 122). Non-fasting and dynamic blood glucose data, weight measurements and immunohistochemistry results indicated that the composition of the local microenvironment surrounding transplanted islets is a factor in promoting their long-term survival and function. In particular, microporous polymer scaffolds fabricated from copolymers of lactide and glycolide were adsorbed with collagen IV, fibronectin, laminin-332 or serum proteins prior to seeding with 125 mouse islets. Islet-seeded scaffolds were then implanted onto the epididymal fat pad of syngeneic mice with streptozotocin-induced diabetes. Non-fasting glucose levels, weight gain, response to glucose challenges, and histology were employed to assess graft function for ten months following transplantation. Mice transplanted with islets seeded onto scaffolds adsorbed with collagen IV achieved euglycemia fastest and the response to glucose challenge was similar to normal mice. Fibronectin and laminin similarly promoted euglycemia, yet required more time than collagen IV and less time than serum. Histopathological assessment of retrieved grafts demonstrated that coating scaffolds with specific extracellular matrix proteins increased the total islet area in the sections and vessel density within the islets, relative to controls. It was shown that extracellular matrix proteins adsorbed to microporous scaffolds enhance the function of transplanted islets, with collagen IV maximizing graft function relative to the other proteins tested. These scaffolds enable the creation of well-defined microenvironments that promote graft efficacy at extrahepatic sites.

Three-dimensional, porous polymer structures (known as scaffolds) are used in tissue engineering applications to create synthetic microenvironments that, for example, promote new tissue formation, and serve as vehicles for delivering transplanted cells to specific sites within the body (Lavik, E & Langer, R. Tissue engineering: current state and perspectives Appl Microbiol Biotechnol 65, 1-8 (2004)). Achieving the formation of desired tissues and promoting the survival and function of transplanted cells requires the ability to direct cellular behavior through the controlled provision of biological signals, such as, for example, soluble growth factors. Thus, the development of drug-releasing scaffolds is of general interest in the field of tissue engineering. In some embodiments, the present invention provides protein and/or DNA releasing scaffolds as a platform for transplanting cells (e.g., as a platform for transplanting pancreatic islet cells (Blomeier, H. et al. Polymer scaffolds as synthetic microenvironments for extrahepatic islet transplantation. Transplantation 82, 452-459 (2006))).

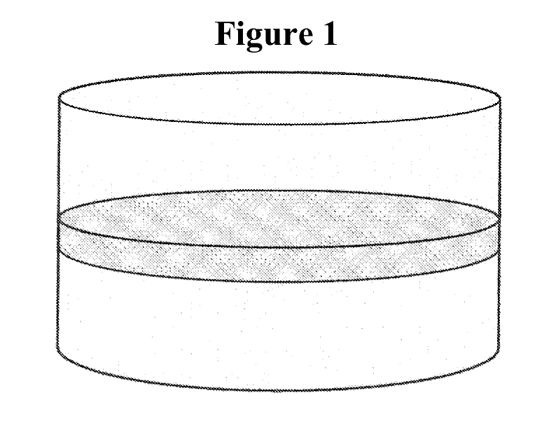

In experiments conducted during the course of development of embodiments for the present invention, a novel scaffold design was developed that exhibits an enhanced capacity for delivery of, for example, pharmaceutical agents, DNA, RNA, and/or biological material (e.g., pancreatic islet cells). In some embodiments, the scaffold design comprises a thin, non-porous center layer that is sandwiched between two highly porous outer layers (see, FIG. 1). In some embodiments, the center layer functions as a drug delivery device, while the outer layers allow for cell-seeding and tissue infiltration. In some embodiments, loading drugs into a non-porous layer minimizes losses during particulate leaching, and slows the release to facilitate sustained delivery. In some embodiments, the outer and inner layers are optimized independently from each other, such that they have entirely different properties (e.g., non-porous versus highly porous). As such, in some embodiments, each layer is designed specifically for a given function so as to optimize performance.

A method for fabricating porous poly(lactide-co-glycolide) (PLG) scaffolds has been previously described (Mooney, D. J., Baldwin, D. F., Suh, N. P., Vacanti, J. P. & Langer, R. Novel approach to fabricate porous sponges of poly(D,L-lactic-co-glycolic acid) without the use of organic solvents. Biomaterials 17, 1417-1422 (1996), herein incorporated by reference in its entirety; Harris, L. D., Kim, B. S. & Mooney, D. J. Open pore biodegradable matrices formed with gas foaming. J Biomed Mater Res 42, 396-402 (1998)), herein incorporated by reference in its entirety, and the ability to deliver proteins and DNA from such scaffolds documented (Richardson, T. P., Peters, M. C., Ennett, A. B. & Mooney, D. J. Polymeric system for dual growth factor delivery. Nat Biotechnol 19, 1029-1034 (2001), herein incorporated by reference in its entirety; Shea, L. D., Smiley, E., Bonadio, J. & Mooney, D. J. DNA delivery from polymer matrices for tissue engineering. Nat Biotechnol 17, 551-554 (1999), herein incorporated by reference in its entirety; Jang, J. H., Rives, C. B., & Shea, L. D. Plasmid delivery in vivo from porous tissue-engineering scaffolds: transgene expression and cellular transfection. Mo Therl 12, 475-483 (2005), herein incorporated by reference in its entirety; Sheridan, M. H., Shea, L. D., Peters, M. C. & Mooney, D. J. Bioabsorbable polymer scaffolds for tissue engineering capable of sustained growth factor delivery. J Control Release 64, 91-102 (2000)), herein incorporated by reference in its entirety. Certain limitations have been encountered with these existing scaffold technologies, namely the potential discrepancy involved in designing a scaffold with an optimal physical structure that simultaneously functions as an effective drug delivery device. In some instances, these two design considerations are not compatible, and it becomes a challenge to fabricate a scaffold that satisfies both design requirements. Accordingly, in some embodiments, the present invention provides a layered scaffold design to overcome such limitations. In some embodiments, the present invention provides a layered scaffold design having layers with different physical properties to serve different functions.

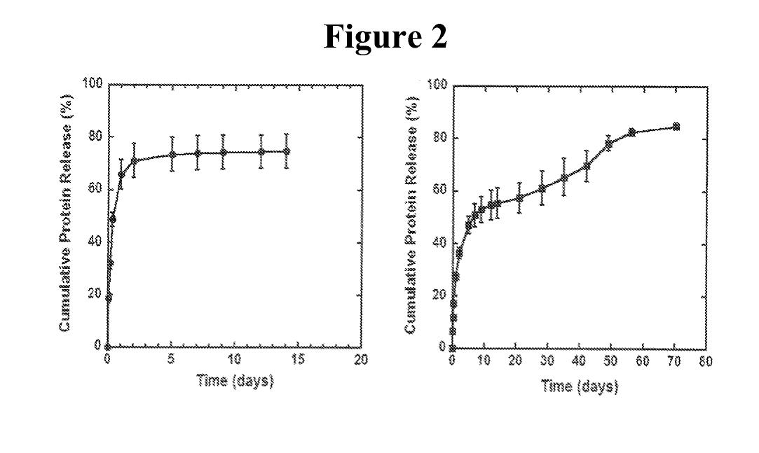

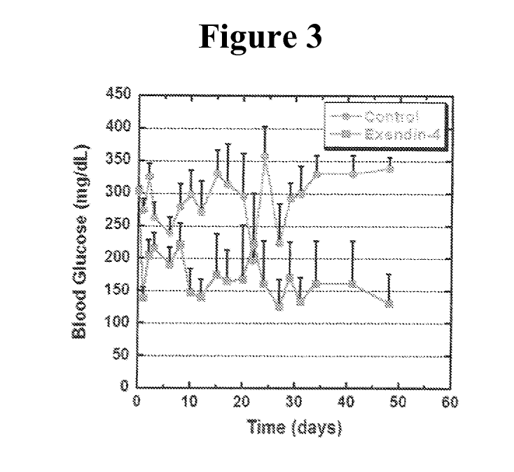

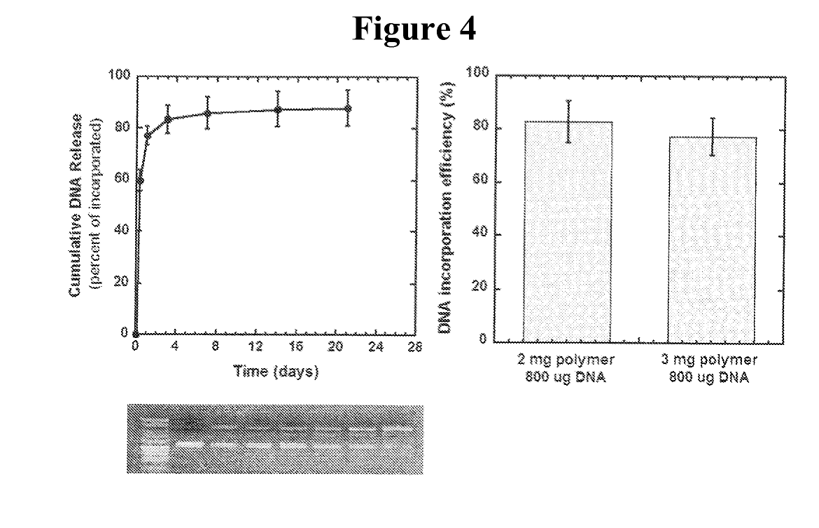

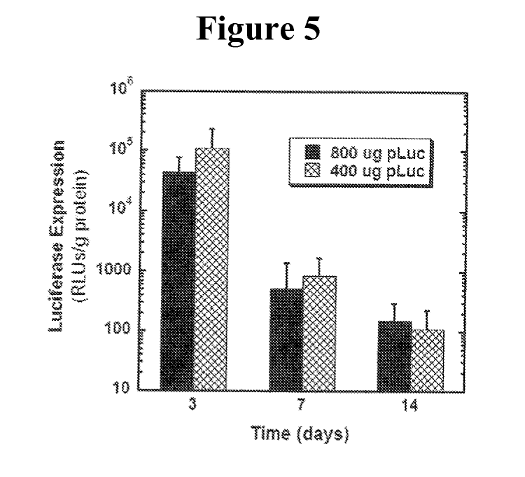

In experiments conducted during the course of development of embodiments for the present invention, the novel scaffold design was used for the delivery of both proteins and DNA. Exendin-4 is a small peptide that exhibits several positive effects on islet cells, including (i) promoting glucose-stimulated insulin secretion, (ii) inhibiting islet cell apoptosis, and (iii) stimulating islet cell proliferation (Ghofaili, K. A. et al. Effect of exenatide on beta cell function after islet transplantation in type 1 diabetes. Transplantation 83, 24.-28 (2007); Sharma, A. et al. Exendin-4 treatment improves metabolic control after rat islet transplantation to athymic mice with streptozotocin-induced diabetes. Diabetologia 49, 1247-1253 (2006); Urusova, I. A., Farilla, L., Hui, H., D'Amico, E. & Perfetti, R. GLP-1 inhibition of pancreatic islet cell apoptosis. Trends Endocrinol Metab 15, 27-33 (2004); Xu, G, Stoffers, D. A., Habener, J. F. & Bonner-Weir, S. Exendin-4 stimulates both beta-cell replication and neogenesis, resulting in increased beta-cell mass and improved glucose tolerance in diabetic rats. Diabetes 48, 2270-2276 (1999); Movassat, J., Beattie, G. M., Lopez, A. D. & Hayek, A. Exendin 4 up-regulates expression of PDX 1 and hastens differentiation and maturation of human fetal pancreatic cells. J Clin Endocrinol Metab 87, 4775-478 1 (2002)). In experiments conducted during the course of development of embodiments for the present invention, the layered scaffold design was shown to achieve sustained delivery of exendin-4 for 2 months (FIG. 2), and demonstrated increased blood glucose control in diabetic mice that were transplanted with islets on exendin-4 releasing scaffolds relative to controls (FIG. 3). In addition, the layered scaffolds were also shown to deliver of DNA, as the design allows for efficient incorporation of large amounts of DNA (FIG. 4). In addition, layered DNA scaffolds that were implanted into the epididymal fat of mice provided detectable levels of transgene expression for 2 weeks (FIG. 5).

Accordingly, in certain embodiments, the present invention provides methods for transplanting pancreatic islet cells or other desired cell types. The methods are not limited to particular manner for transplanting pancreatic islet cells. In some embodiments, the methods comprise implanting scaffolds having thereon pancreatic islet cells into a subject (e.g., a human, a mouse, a cat). The methods are not limited to a particular type of scaffold. In some embodiments, the scaffold is a microporous scaffold. In some embodiments, the microporous scaffold is a PLG microporous scaffold. In some embodiments, the scaffold has thereon extracellular matrix (ECM) proteins. In some embodiments, the ECM proteins include, but are not limited to, collagen IV, fibronectin, and/or laminin. In some embodiments, the scaffold has thereon exendin-4. In some embodiments, the scaffold has thereon DNA, RNA, etc. In some embodiments, the scaffold has a thin nonporous layer positioned between two highly porous outer layers. In some embodiments, the nonporous layer has thereon pharmaceutical agents, DNA, RNA, ECM proteins, exendin-4, etc. In some embodiments, the methods are used for treating type 1 diabetes in a subject (e.g., increased blood glucose control; restoring euglycemia). In certain embodiments, the present invention provides microporous scaffolds having thereon pancreatic islet cells. In some embodiments, the microporous scaffolds additionally have thereon ECM proteins and/or exendin-4.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject, unless indicated otherwise.

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function. Test compounds comprise both known and potential therapeutic compounds. A test compound can be determined to be therapeutic by screening using the screening methods of the present invention. In some embodiments of the present invention, test compounds include antisense compounds.

As used herein, the term “sample” is used in its broadest sense. In one sense, it is meant to include a specimen or culture obtained from any source, as well as biological and environmental samples. Biological samples may be obtained from animals (including humans) and refers to a biological material or compositions found therein, including, but not limited to, bone marrow, blood, serum, platelet, plasma, interstitial fluid, urine, cerebrospinal fluid, nucleic acid, DNA, tissue, and purified or filtered forms thereof. Environmental samples include environmental material such as surface matter, soil, water, and industrial samples. Such examples are not however to be construed as limiting the sample types applicable to the present invention.

As used herein, the term “effective amount” refers to the amount of a composition sufficient to effect beneficial or desired results. An effective amount can be administered in one or more administrations, applications or dosages and is not intended to be limited to a particular formulation or administration route.

As used herein, the term “administration” refers to the act of giving a drug, prodrug, or other agent, or therapeutic treatment (e.g., compositions of the present invention) to a subject (e.g., a subject or in vivo, in vitro, or ex vivo cells, tissues, and organs). Exemplary routes of administration to the human body can be through the eyes (ophthalmic), mouth (oral), skin (transdermal), nose (nasal), lungs (inhalant), oral mucosa (buccal), ear, rectal, by injection (e.g., intravenously, subcutaneously, intratumorally, intraperitoneally, etc.), by surgical implantation, and the like.

As used herein, the terms “co-administration” and “co-administer” refer to the administration of at least two agent(s) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s).

As used herein, the term “pharmaceutical composition” refers to the combination of an active agent with a carrier, inert or active, making the composition especially suitable for diagnostic or therapeutic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintigrants (e.g., potato starch or sodium starch glycolate), and the like. The compositions also can include stabilizers and preservatives. For examples of carriers, stabilizers and adjuvants. (See e.g., Martin, Remington's Pharmaceutical Sciences, 15th Ed., Mack Publ. Co., Easton, Pa. (1975), incorporated herein by reference).

As used herein, the term “pharmaceutically acceptable salt” refers to any salt (e.g., obtained by reaction with an acid or a base) of a compound of the present invention that is physiologically tolerated in the target subject (e.g., a mammalian subject, and/or in vivo or ex vivo, cells, tissues, or organs). “Salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts. Examples of bases include, but are not limited to, alkali metal (e.g., sodium) hydroxides, alkaline earth metal (e.g., magnesium) hydroxides, ammonia, and compounds of formula NW.sub.4.sup.+, wherein W is C.sub.1-4 alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, chloride, bromide, iodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation. For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

### EXAMPLES

The following examples are provided in order to demonstrate and further illustrate certain preferred embodiments and aspects of the present invention and are not to be construed as limiting the scope thereof.

### Example 1

### Materials and Methods

### Fabrication of Microporous Scaffolds

PLG microspheres were made as previously described (Jang J H, Shea L D. Controllable delivery of non-viral DNA from porous scaffolds. J Control Release 2003; 86 (1): 157) using a single emulsion/solvent evaporation process and used as building blocks for scaffold fabrication. PLG (75:25 molar ratio of D,L-lactide to glycolide, i.v.=0.6-0.8 dL/g) (Alkermes, Cincinnati, Ohio) was dissolved in methylene chloride to make a 2% (w/v) solution. This solution was emulsified in an aqueous 1% (w/v) poly(vinyl alcohol) (PVA, 88% hydrolyzed, average MW 22,000) (Acros Organics, Fair Lawn, N.J.) solution by homogenization at 7,000 rpm for 45 seconds. This homogenized solution was diluted in deionized (DI) water and stirred for 3 hours at room temperature to evaporate the organic solvent. Microspheres were collected by centrifugation (4,000 rpm for 10 minutes), washed three times with DI water to remove residual PVA, lyophilized to form a powder and stored in a vacuum desiccator until use.

Microporous scaffolds were fabricated using a previously described gas foaming/particular leaching process (Jang J H, Shea L D. Controllable delivery of non-viral DNA from porous scaffolds. J Control Release 2003; 86 (1): 157). Briefly, 7 mg of PLG microspheres were mixed with 190 mg of sodium chloride (NaCl) crystals (250 μm<diameter<425 μm), loaded into a cylindrical stainless steel die (internal diameter 5 mm), and compression molded at 1500 psi for 30 seconds using a Carver laboratory press (Carver, Muncie, Ind.). The compressed pellets were then incubated with 95% humidity at 37° C. for 24 h to fuse the salt crystals in order to create an interconnected internal pore structure. After incubation, the mixture was dried under vacuum and equilibrated with CO₂ (800 psi) for 16 h in a custom-made pressure vessel. Rapid release of CO₂ caused the polymer microspheres to expand and fuse into a continuous matrix (Jang J H, Shea L D. Controllable delivery of non-viral DNA from porous scaffolds. J Control Release 2003; 86 (1): 157). The fused constructs were immersed in an excess of water for 4 h to leach the salt, dried overnight and stored in a vacuum desiccator until use.

### Protein Adsorption to Scaffolds

Scaffolds were treated in the manner described below on the day prior to islet isolation and seeding. For protein adsorption, dry scaffolds were immersed in 0.5 N NaOH for 1 minute (Park G E, Pattison M A, Park K, Webster T J. Accelerated chondrocyte functions on NaOH-treated PLGA scaffolds. Biomaterials 2005; 26 (16): 3075) followed by immersion in an excess of water (washed until pH was neutral). Scaffolds were dried for 5 minutes at room temperature before placing in 70% EtOH for 1 min. Scaffolds were again dried for 5 minutes before being placed into individual wells of a 24-well tissue culture dish. Collagen IV (50 μL at 1 mg/ml; Sigma), fibronectin (50 μL at 1 mg/ml; Sigma), laminin-332 (formerly termed laminin-5 and hereafter referred to as “laminin”; 50 μL of conditioned cell culture media from 804G cells containing approximately 1 mg/ml of laminin-332 (Baker S E, DiPasquale A P, Stock E L, Quaranta V, Fitchmun M, Jones J C. Morphogenetic effects of soluble laminin-5 on cultured epithelial cells and tissue explants. Exp Cell Res 1996; 228 (2): 262)), or serum-containing media [RPMI-1640 media (Gibco-BRL, Grand Island, N.Y.) supplemented with 10% heat-inactivated fetal calf serum (Hyclone, Logan, Utah), 100 U/ml penicillin-G, 100 mg/ml streptomycin sulfate, and 1 mmol/l L-glutamine; hereafter referred to as “SCM”] were added to the scaffold and incubated at room temperature for two hours, followed by addition of 50 μL of the same component to each scaffold. Scaffolds were then incubated with 95% humidity at 37° C. overnight to allow for protein adsorption. Prior to islet seeding, 100 μl of fresh SCM was applied to the top of each scaffold.

Protein adsorption to the scaffold surface was assessed using the picrosirius stain (Junqueira L C, Bignolas G, Brentani R R. Picrosirius staining plus polarization microscopy, a specific method for collagen detection in tissue sections. Histochem J 1979; 11 (4): 447). Following overnight incubation, scaffolds were washed three times with PBS to wash away any unbound protein and placed in a new 24-well culture dish. After staining, scaffolds were visualized by light microscopy to identify adsorbed proteins.

### Animals and Induction of Diabetes

Male C57BL/6 mice (Jackson Laboratories, Bar Harbor, Me.) between 8 and 12 weeks of age were used as islet donors and transplant recipients. Four days prior to islet transplantation, graft recipient mice were injected intraperitoneally with 220 mg/kg of streptozotocin (Sigma, St. Louis, Mo.) to chemically induce irreversible diabetes (Dufrane D, van Steenberghe M, Guiot Y, Goebbels R M, Saliez A, Gianello P. Streptozotocin-induced diabetes in large animals (pigs/primates): role of GLUT2 transporter and beta-cell plasticity. Transplantation 2006; 81 (1): 36). Non-fasting blood glucose levels were measured in whole blood samples obtained from the tail of the animals using a One Touch Basic glucose monitor (Lifescan, Milpitas, Calif.). Mice were used in these studies only if they had blood glucose measurements greater than 300 mg/dL on consecutive days prior to transplantation. The blood glucose levels of donor mice were also checked prior to islet isolation to verify that they were metabolically normal.

### Islet Isolation, Scaffold Seeding and Transplantation

Islet isolation and scaffold seeding were performed as previously described except that each recipient only received 125 islets (Blomeier H, Zhang X, Rives C, et al. Polymer scaffolds as synthetic microenvironments for extrahepatic islet transplantation. Transplantation 2006; 82 (4): 452). Islets were isolated from donor pancreata by a mechanically-enhanced enzymatic digestion using collagenase (type XI; Sigma). Donor mice were anesthetized with an intraperitoneal injection of 250 mg/kg tribromoethanol (Avertin; Fluka Chemical, Buchs, Switzerland). After a midline abdominal incision, the common bile duct was cannulated and injected with a cold solution of collagenase in Hank's balanced salt solution (HBSS). The pancreas was dissected, removed and digested at 37° C. for 15 minutes. After filtration through a mesh screen, the filtrate was applied to a discontinuous dextran (Sigma) gradient. Islets were hand-picked and counted under microscopic guidance. Islets were seeded onto each scaffold in a minimal volume of media by applying them to the scaffold and allowing them to filter into the microporous structure. Examination of the tissue culture media following removal of the scaffolds demonstrated that greater than 95% of the islets stayed on the scaffolds following seeding. Scaffolds were then incubated at 37° C. in 5% CO₂ and 95% air for 30 min. At that time, 20 μL of SCM was added to the top of each scaffold and returned to the incubator. After 60 min incubation, 5 mL of SCM was added to the tissue culture well in which each scaffold was placed and returned to the 37° C. incubator for 30 min prior to transplantation.

Recipient mice were anesthetized with an intraperitoneal injection of Avertin (250 mg/kg body wt) and the abdominal region was shaved and prepped in a sterile manner. Following a short, midline lower abdominal incision, the right epididymal fat pad was identified and spread on the shaved, exterior abdominal surface. Scaffolds pre-seeded with islets were then placed on and wrapped by the epididymal fat pad and returned to the intraperitoneal cavity. Scaffolds not seeded with islets but incubated overnight in SCM were transplanted as negative controls. The wound was closed in two layers. Mice were allowed free access to food and water post-operatively and were routinely checked throughout the duration of the study for any signs of infection around the surgical site.

### Assessment of Graft Function

Following transplantation, non-fasting blood glucose measurements were taken between 12:00 and 17:00 as described above using the following schedule: everyday during the first post-operative week, every other day during weeks 2-5, once per week during weeks 6-25, and once per month thereafter until the conclusion of the study. Grafts were considered to be functional if glucose levels were maintained at less than 200 mg/dL and mice did not reconvert to a hyperglycemic state for the duration of the study. Following graft removal at the end of post-operative week 42, blood glucose levels were monitored for 72 hours, at which time the mice were sacrificed.

Intraperitoneal glucose tolerance tests (IPGTTs) were performed at four and forty weeks following transplantation in order to assess the grafts' ability to respond to glucose challenges. Following a 6 hour fast, 2 g/kg of 50% dextrose (Abbott Labs, North Chicago, Ill.) was injected intraperitoneally. Blood glucose levels were measured at baseline (prior to injection), 15, 30, 60 and 120 min after glucose injection. Area under the curve (AUC) for each animal was calculated using the trapezoidal rule (Cheung B W, Cartier L L, Russlie H Q, Sawchuk R J. The application of sample pooling methods for determining AUC, AUMC and mean residence times in pharmacokinetic studies. Fundam Clin Pharmacol 2005; 19 (3): 347). The area corresponding to the baseline glucose measurement multiplied by 120 minutes was subtracted from the total AUC calculated in order to account for any baseline differences between the animals.

### Histological Analysis

Histological analysis was performed to characterize the morphology of transplanted islets and to quantify islet area and vascular density within the islet. On post-operative day 7 or 297, fat pads containing the islet grafts were explanted and fixed in 4% paraformaldehyde. Fixed specimens were embedded in paraffin or Tissue-Tek O.C.T. compound (Miles Scientific, Elkhart, Ind.), and 5 μm paraffin or 10 μm cryosections were prepared, respectively. Immunohistochemistry was performed to confirm the presence of beta-cells using guinea pig anti-insulin antibody (1:100; Zymed, South San Francisco, Calif.) and a biotinylated goat anti-guinea pig immunoglobulin (1:1000; Vector, Burlingame, Calif.), followed by streptavidin-horseradish peroxidase which was revealed by staining with 3,3′-diaminobenzidine (DAB). Sections were counter-stained with hematoxylin. Paraffin sections were also stained with hematoxylin-eosin according to standard protocols. Digital images were acquired using a Spot camera via the accompanying image analysis software (Diagnostic Instruments, Inc., Sterling Heights, Mich.) attached to a Nikon Eclipse 50i microscope (Nikon, Tokyo, Japan).

### Quantification of Islet Size and Vascular Density

Assessment of islet size and vascular density was performed in grafts removed after 297 days of implantation. For each condition, three randomly chosen paraffin-embedded grafts were serially sectioned as described above. Note that for the serum condition, the grafts used were from animals whose diabetes had been reversed. The first section containing insulin positive cells was identified and labeled “base section.” Starting at 50 μm after the base section and then at approximately 60 μm intervals thereafter, slides were selected for insulin-IHC and H&E staining Five slides per tissue sample per condition were collected in this manner, each set representing a depth within the scaffold of approximately 300 μm. Few islets were observed at greater depths within the graft. One section on each slide was stained for insulin while the other was stained using hematoxylin-eosin. The section stained for insulin was used for verification of islet location, while the H&E section was used for identification of blood vessels. Pictures were taken using a 40× objective as described above and assembled into composite images in Adobe Photoshop CS3 Extended (Adobe Systems Inc., San Jose, Calif.). Using Photoshop, the area of each islet was measured and the corresponding number of intraislet vessels was counted after blinding the observer to the condition being evaluated.

### Statistical Analysis

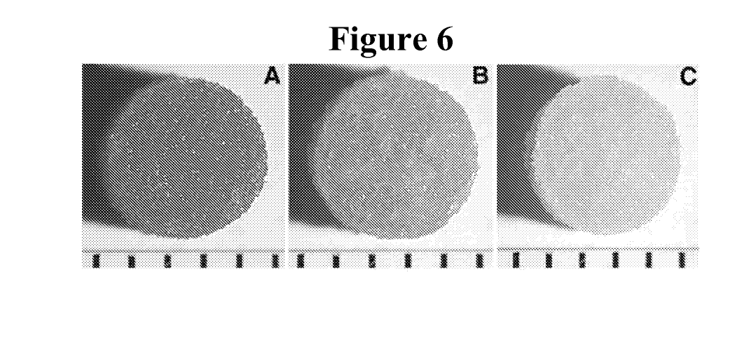

All values are reported as the mean±SEM. Differences in the number of days to reach euglycemia between experimental groups were compared using the Kaplan-Meier survival analysis and the Log-Rank test. Statistical analyses for comparison of weight and IPGTT data, and all bar graphs in FIG. 6, were performed by using Student's t test. A P-value of less than 0.05 was considered statistically significant.

### Results

### Protein Adsorption to Scaffolds

Protein adsorption was visualized to determine an appropriate protein concentration and duration of incubation that would provide a homogeneous distribution throughout the scaffold. Hydrolyzed-scaffolds incubated with collagen IV (FIG. 6A) demonstrated extensive protein adsorption throughout the scaffold, whereas non-hydrolyzed scaffolds (FIG. 6B) demonstrated a lower staining intensity as well as an inconsistent distribution of staining in the scaffold. Hydrolyzed-scaffolds incubated in PBS had no staining (FIG. 6C). Increasing the concentration of collagen IV from 0.00 to 3.71 mg/ml increased the intensity of staining, as did increasing the time of incubation from 1 hour to 16 hours. Examination of scaffold cross-sections following staining confirmed that protein adsorption was homogenous throughout the entire scaffold volume. These experiments were repeated using fibronectin and laminin with similar results. Based on these results, overnight incubation of hydrolyzed scaffolds in 1 mg/ml of the selected ECM component was employed in all subsequent studies.

### Specific ECM Proteins Improve Islet Function Following Transplantation

Subsequent experiments investigated the ability of collagen IV, fibronectin, and laminin—ECM proteins known to be present in pancreatic islets in vivo—to enhance islet function following transplantation. In addition to transplanting islets onto scaffolds coated with collagen IV, fibronectin and laminin, a fourth group of mice was transplanted with scaffolds that had been incubated in serum-containing media (SCM) prior to islet seeding. As a negative control, a fifth group of mice was implanted with scaffolds that had been incubated in SCM but not seeded with islets prior to implantation. In these studies, a syngeneic animal model was used, which allowed for investigation of the impact of various ECM components on graft success without complicating effects from immunosuppressive agents.

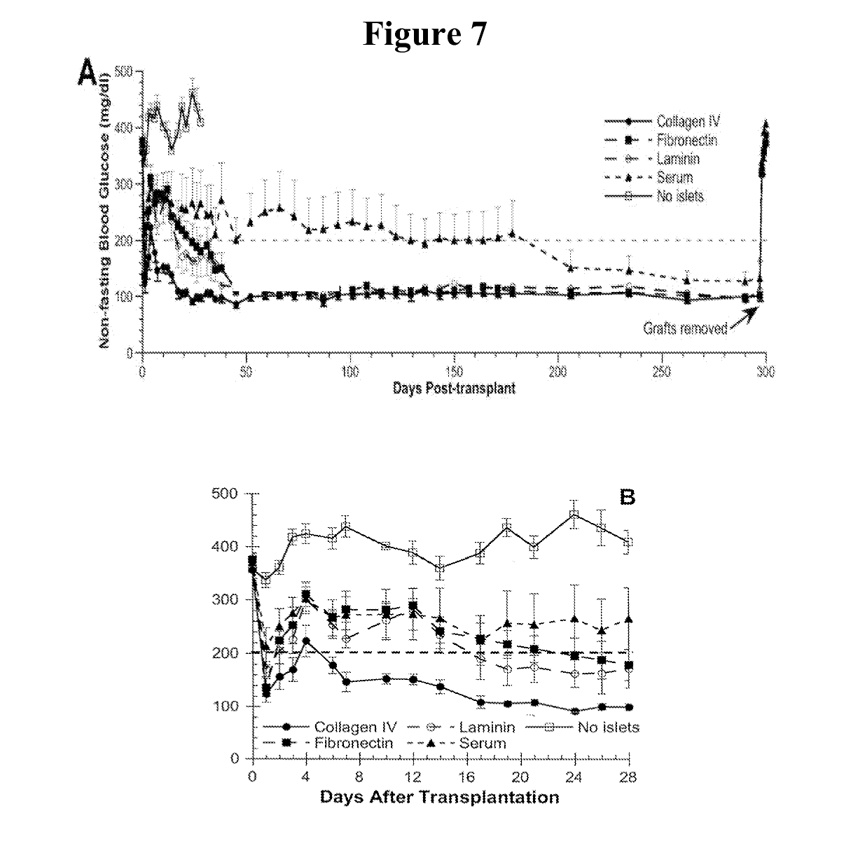

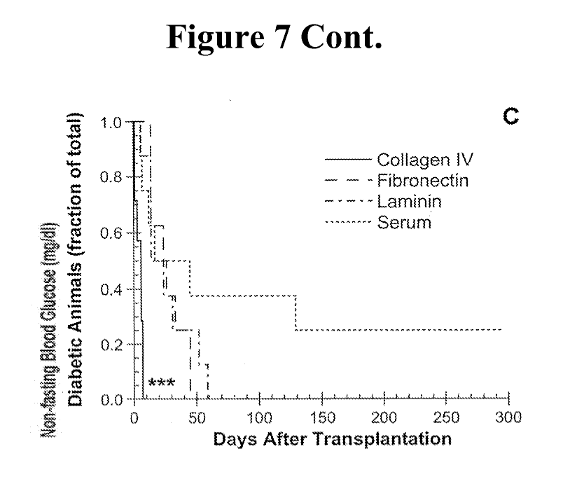

Mice transplanted with scaffolds pre-adsorbed with collagen IV achieved euglycemia most rapidly, with a mean time to euglycemia of 4.4±1.0 days (100% converted; n=7), compared to 26.9±4.6 days (100% converted; n=8) for the fibronectin group, 26.8±6.8 days for the laminin group (100% converted; n=8) and 36.0±18.1 days (75% converted; n=8) for the serum group (FIGS. 7A and B). Mice implanted with scaffolds lacking islets (n=8) remained hyperglycemic with glucose levels between 282 and 547 mg/dl before being sacrificed on day 28. All other mice were maintained until day 297 post-transplantation, at which time the fat pad containing the graft was removed from each animal. In all cases, euglycemic animals reverted to a state of hyperglycemia within 24 hr after scaffold removal, confirming that the islets contained within the fat pad were responsible for sustaining euglycemia (FIG. 7A). The time to euglycemia for the collagen IV group was significantly less than that of the other groups as determined by the log-rank test applied to a Kaplan-Meier survival curve (P<0.001 for collagen IV vs. fibronectin, laminin and serum) (FIG. 7C). None of the other pair-wise comparisons had significance at the P=0.05 level.

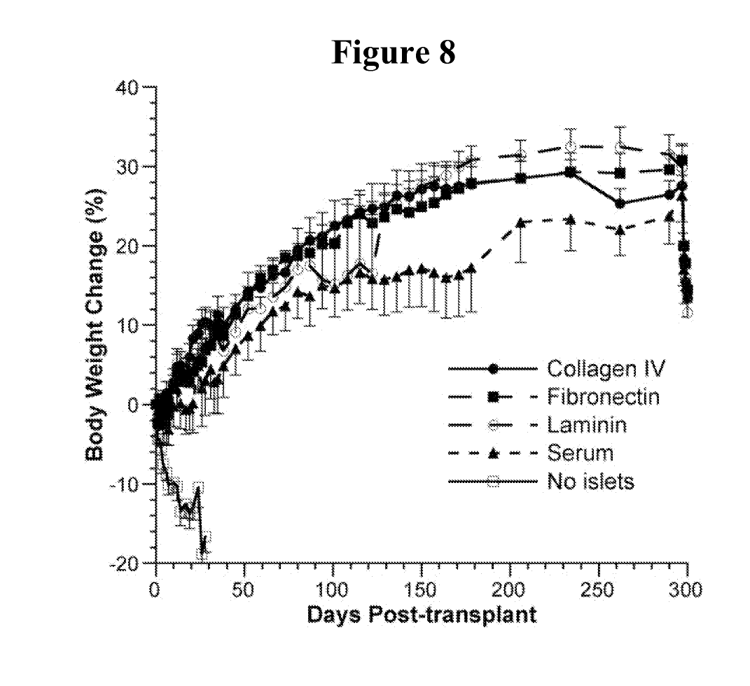

Consistent with the blood glucose levels, mice transplanted with islets exhibited similar changes in body weight from day 0 (day of transplant) to day 297 [27.6±1.3% for the collagen IV group, 30.8±2.1% for the fibronectin group, 29.9±2.7% for the laminin group, and 26.3±3.3% for the serum group] (FIG. 8). While the serum group consistently exhibited a lower percent change in weight compared to the three experimental groups, these differences were not statistically significant at any time point as determined by Student's t test (P>0.05). Mice in the negative control group lost an average 16.6±2.0% of their body weight before being sacrificed on day 28.

### Specific ECM Proteins Improve Islet Response to Glucose Challenges

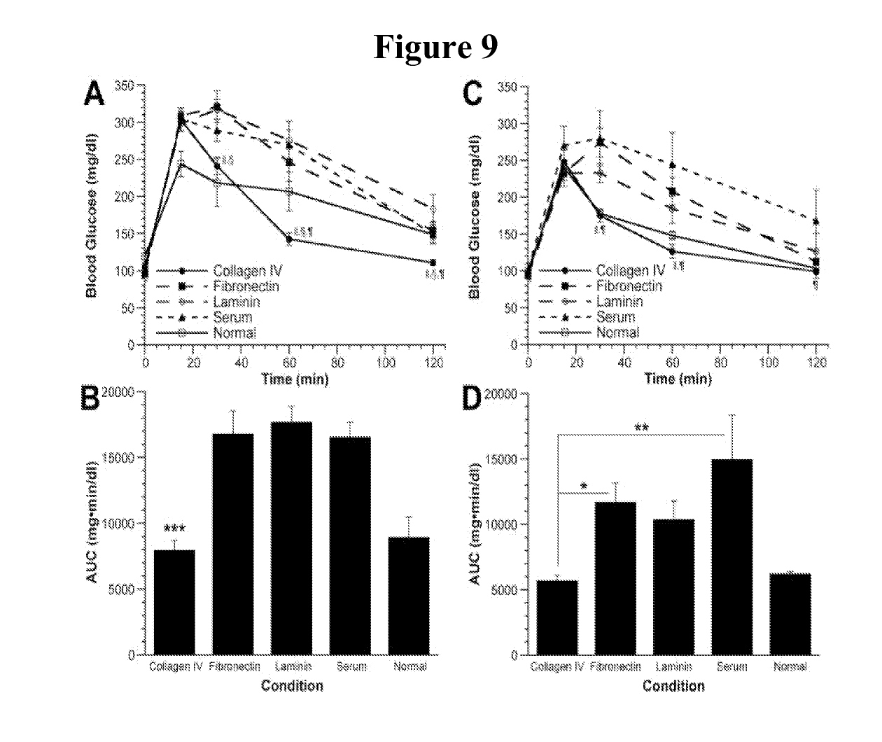

To further investigate the connection between ECM proteins and islet function, intraperitoneal glucose tolerance tests (IPGTT) were performed at four and forty weeks post-transplant on mice in which euglycemia had been restored. For comparison, an IPGTT was also performed on non-diabetic, age-matched C57BL/6 mice (n=3) at both time points. At four weeks post-transplant, baseline fasting glucose levels were similar between the five groups of mice (FIG. 9A). At 30 min, however, glucose levels in the collagen IV and normal groups were significantly lower than in the fibronectin and laminin groups. Similarly, at 60 min, glucose levels in the collagen IV group were significantly lower than the laminin, fibronectin and serum groups. At 120 min, glucose levels had returned to near baseline for all groups except for the laminin group, which was significantly higher than the collagen IV group. Glucose levels in the collagen IV and normal groups were similar at all time points. The area under the curve (AUC) for the collagen IV group was similar to that of the normal control mice but significantly less (P<0.001) than that of the other three treatment groups (FIG. 9B).

Significant differences between groups were also found at forty weeks post-transplant (FIG. 9C). Glucose levels in the collagen IV and normal groups were significantly lower than the fibronectin and serum groups 30 minutes following glucose injection. Again, at 60 minutes, the collagen IV group had glucose levels significantly lower than the serum group. At forty weeks post-transplant, the AUC for the collagen IV group (FIG. 9D) was similar to the normal group but significantly less than the serum group (P<0.01).

### ECM Proteins Support Islet Architecture and Enhance Total Islet Mass Post-Transplant

Islets seeded onto scaffolds coated with collagen IV were found to maintain normal cell-cell interactions and intact islet architecture, which may be necessary for islet function, when removed for histological analysis 7 days after implantation. The periphery of islets is in direct contact with the protein-coated scaffold surface on which they sit. Additionally, all islets were found to be located within a distance of approximately 400 μm from the surface on which they were seeded. Similar results were seen using fibronectin- and laminin-coated scaffolds; however, islet architecture appeared markedly disrupted—whereby normal cell-cell interactions were lost and individual insulin-positive cells were seen strewn over the scaffold surface and within its interior, when seeded onto serum-coated scaffolds.

The architecture and size of transplanted islets were assessed for islet grafts explanted 297 days after transplantation. Immunohistochemical analyses performed on tissue sections from the three experimental conditions revealed large numbers of insulin-positive cells arranged in well circumscribed and highly vascularized structures. Although immunostaining for insulin was present in sections from the serum-coated scaffolds, islet morphology was different and total islet area in this group was markedly smaller relative to the other groups in all sections observed. For all conditions, no scaffold material remained visible in the grafts, indicating that the polymer had degraded and that transplanted islets had become well integrated with the host tissue. Additionally, an abundance of larger vessels and periislet vessels were observed next to and around the islets in all experimental conditions whereas few to none were observed in the serum condition.

### Quantification of Islet Area and Vascular Density

Based on the observation that tissue sections from the ECM conditions appeared to contain more islet mass than the serum controls, these differences were quantitatively assessed. As described in the Materials and Methods section, Photoshop was used to calculate the area of individual islets and count the number of intraislet blood vessels. By summing the areas of individual islets in a given tissue section and dividing by the number of sections counted per condition, the average total islet area per section was calculated. While the three ECM conditions all had, on average, significantly more islet area per section than the serum condition (FIG. 6B; P<0.05), average islet size between groups was not significantly different.

Vessel density was also assessed and revealed that the three ECM groups had significantly more intraislet microvessels than the serum group (P<0.001). Using the area data calculated above, vascular density (vessels/mm²) was also calculated and showed that while the collagen IV and fibronectin groups had similar mean vascular density, both were significantly higher than the laminin and serum groups (P<0.001). The vascular density of the laminin group was also found to be significantly higher than the serum group (P<0.001). It is interesting to note that the calculated vascular densities for islets seeded onto scaffolds coated with collagen IV (1484±27 vessels/mm²) and fibronectin (1455±28 vessels/mm²) are similar to those previously reported for native C57BL/6 islets but significantly more than the vascular density found in islets transplanted beneath the kidney capsule (Mattsson G, Jansson L, Nordin A, Carlsson P O. Impaired revascularization of transplanted mouse pancreatic islets is chronic and glucose-independent. Transplantation 2003; 75 (5): 736). The presence and distribution of blood vessels within and around transplanted islets is a requirement for their survival and function and is consistent with the results seen in the 40-week IPGTT studies.

In experiments conducted during the course of development for embodiments of the present invention, it was demonstrated that ECM components significantly improved the efficacy of islet grafts in an animal model of T1DM. The observed effect of ECM components on the restoration of euglycemia could be mediated by interactions between the adsorbed proteins and islets, between proteins and the host tissue, or a combination of the two. Previous reports have shown that ECM components interact with a variety of cell-surface integrins to affect intracellular processes such as beta-cell survival (Hammar E, Parnaud G, Bosco D, et al. Extracellular matrix protects pancreatic beta-cells against apoptosis: role of short- and long-term signaling pathways. Diabetes 2004; 53 (8): 2034), differentiation (Jiang F X, Harrison L C. Extracellular signals and pancreatic beta-cell development: a brief review. Mol Med 2002; 8 (12): 763), proliferation (Hayek A, Lopez A D, Beattie G M. Enhancement of pancreatic islet cell monolayer growth by endothelial cell matrix and insulin. In Vitro Cell Dev Biol 1989; 25 (2): 146) and insulin secretion (Bosco D, Meda P, Halban P A, Rouiller D G. Importance of cell-matrix interactions in rat islet beta-cell secretion in vitro: role of alpha6beta1 integrin. Diabetes 2000; 49 (2): 233). These in vitro findings establish, for example, the importance of integrin-mediated signaling on islet function, and the experiments conducted during the course of development for embodiments of the present demonstrate that ECM components significantly enhance the function of transplanted islets in an animal model of T1DM. Interestingly, whereas it was found that collagen IV has a markedly positive impact on the function of transplanted islets, Kaido et al. reported that islets cultured on collagen IV-coated tissue culture wells showed marked suppression of insulin gene transcription and significant glucose-independent insulin secretion (Kaido T, Yebra M, Cirulli V, Rhodes C, Diaferia G, Montgomery A M. Impact of defined matrix interactions on insulin production by cultured human beta-cells: effect on insulin content, secretion, and gene transcription. Diabetes 2006; 55 (10): 2723). A difference between these two approaches is that scaffolds provide islets with a 3-D matrix that supports and maintains the architecture and cellular organization found in native islets (Blomeier H, Zhang X, Rives C, et al. Polymer scaffolds as synthetic microenvironments for extrahepatic islet transplantation. Transplantation 2006; 82 (4): 452), whereas in vitro cultured islets gradually transition from spheroidal aggregates to monolayers (Kaido T, Yebra M, Cirulli V, Rhodes C, Diaferia G, Montgomery A M. Impact of defined matrix interactions on insulin production by cultured human beta-cells: effect on insulin content, secretion, and gene transcription. Diabetes 2006; 55 (10): 2723). This beneficial effect of ECM proteins might be mediated by increased adhesive properties of ECM-adsorbed scaffolds, which could act to maintain the native architecture of islets and prevent them from escaping during or after transplantation, although demonstrated that islets seeded onto control scaffolds remained associated with the scaffold following transplantation (Blomeier H, Zhang X, Rives C, et al. Polymer scaffolds as synthetic microenvironments for extrahepatic islet transplantation. Transplantation 2006; 82 (4): 452). This disruption of islet architecture may interfere with integrin-mediated signaling and paracrine interactions between islet cells (Cabrera O, Berman D M, Kenyon N S, Ricordi C, Berggren P O, Caicedo A. The unique cytoarchitecture of human pancreatic islets has implications for islet cell function. Proc Natl Acad Sci USA 2006; 103 (7): 2334). Additionally, Kaido et al. used adult human islets harvested from older donors (45-56 years old)—a factor known to negatively correlate with isolated islet function (Ihm S H, Matsumoto I, Sawada T, et al. Effect of donor age on function of isolated human islets. Diabetes 2006; 55 (5): 1361). Finally, the expansion of the Kaido et al. primary islet cultures for 3-4 days prior to seeding on collagen IV-coated wells complicates a direct comparison, as significant islet cell apoptosis ensues 24-48 hours after isolation with in vitro cultured islets (Thomas F T, Contreras J L, Bilbao G, Ricordi C, Curiel D, Thomas J M. Anoikis, extracellular matrix, and apoptosis factors in isolated cell transplantation. Surgery 1999; 126 (2): 299).

Alternatively, adsorbed proteins may promote the infiltration of host cells, such as endothelial cells, into the scaffold (Rucker M, Laschke M W, Junker D, et al. Angiogenic and inflammatory response to biodegradable scaffolds in dorsal skinfold chambers of mice. Biomaterials 2006; 27 (29): 5027), which interact with the grafted tissue. Endothelial cell infiltration promotes engraftment and revascularization of transplanted islets, which is essential to promoting their survival and function (Olsson R, Maxhuni A, Carlsson P O. Revascularization of transplanted pancreatic islets following culture with stimulators of angiogenesis. Transplantation 2006; 82 (3): 340), which provide an explanation for the significantly increased total islet area in the ECM conditions relative to controls. Islets seeded on control scaffolds may have lacked adequate perfusion and been unable to support their cells' metabolic needs leading to cell death. Enhanced graft revascularization may have also contributed to a better response to glucose during the IPGTT, although, since the IPGTT results for the collagen IV condition were better than the other ECM conditions despite having a similar vascular density as the fibronectin condition, other mechanisms in addition to revascularization may have also contributed to enhanced islet engraftment and function. Thus, adsorbed proteins may exert their effects directly on endothelial cells to promote their infiltration into the scaffold (Tian B, Li Y, Ji X N, et al. Basement membrane proteins play an active role in the invasive process of human hepatocellular carcinoma cells with high metastasis potential. J Cancer Res Clin Oncol 2005; 131 (2): 80). The beneficial effects of the ECM proteins could have also been mediated through interactions with integrins which could promote islet cell survival and proliferation resulting in increased numbers of functioning beta-cells. This interaction could also lead to an increase in the local concentration of VEGF-A (Lai Y, Schneider D, Kidszun A, et al. Vascular endothelial growth factor increases functional beta-cell mass by improvement of angiogenesis of isolated human and murine pancreatic islets. Transplantation 2005; 79 (11): 1530), which would stimulate both infiltration of host endothelial cells and expansion of donor intraislet endothelial cells. Therefore, the combination of direct and indirect effects of matrix components on transplanted islets could explain the observed improvement in outcome when islets were seeded on scaffolds adsorbed with ECM components.

In conclusion, experiments conducted during the development of embodiments for the present invention demonstrated that the presence of ECM proteins on microporous scaffolds leads to a pronounced decrease in the time required to reverse diabetes in C57BL/6 mice relative to non-coated scaffolds. The approach is based on modification of the microenvironment surrounding islets to promote graft survival and function as well as to enhance integration with the recipient. Of the ECM components investigated, the provision of collagen IV was most effective at rapidly reversing STZ-induced hyperglycemia in this animal model. This finding shows that the composition of the islet microenvironment plays an important role in mediating the survival and function of transplanted islets. The scaffold provides a means to manipulate this environment and can be designed to support islet engraftment, and represents a significant departure from previous approaches in which biomaterials have been used for immunoisolation. Moreover, the ability to achieve euglycemia in so short a time with a single transplant of 125 islets (the average islet yield per pancreas is approximately 200) represents the successful application of a single-donor/single-recipient model of islet transplantation—a benchmark that should be routinely achieved in human trials before clinical islet transplantation becomes widely practiced.

### Example II

### Materials and Methods

### Fabrication of DNA-Loaded Scaffolds

DNA-loaded scaffolds were fabricated using a previously described gas foaming/particulate leaching process (Mooney, D. J., Baldwin, D. F., Suh, N. P., Vacanti, J. P. & Langer, R. Novel approach to fabricate porous sponges of poly(D,L-lactic-co-glycolic acid) without the use of organic solvents. Biomaterials 17, 1417-1422 (1996); Harris, L. D., Kim, B. S. & Mooney, D. J. Open pore biodegradable matrices formed with gas foaming. J Biomed Mater Res 42, 396-402 (1998)), although the new layered scaffold design was implemented. PLG (75% D,L lactide/25% glycolide, i.v.=0 76 dl/g) was dissolved in dichloromethane to make either a 2% (w/w) or 6% (w/w) solution, which was then emulsified in 1% poly(vinyl alcohol) to create microspheres The scaffold outer layers were constructed by mixing 1.5 mg of 6% PLG microspheres with 50 mg of NaCl (250-425 μm), and then compressing the mixture in a 5 mm KBr die at 1500 psi using a Carver press. To make the center layer, 2 mg of 2% PLG microspheres were reconstituted in a solution containing plasmid DNA (400 or 800 μg) and lactose (1 mg), and then lyophilized. This lyophilized product was then sandwiched between two outer-layers and compressed at 200 psi. The composite scaffold was then equilibrated with high pressure CO2 gas (800 psi) for 16 hrs in a custom made pressure vessel. Afterwards, the pressure was rapidly released over a period of 25 minutes, which serves to fuse adjacent microspheres creating a continuous polymer structure. To remove the salt, each scaffold was leached in 4 mL of water for 2.5 hours while shaking at 110 rpm, with fresh water replacement after 2 hours.

### Characterization of DNA Incorporation and Release.

The DNA incorporation efficiency is defined as the mass of DNA left in the scaffold after the leaching step divided by the mass of DNA initially input. After leaching, scaffolds were dissolved in chloroform (600 μL) and the DNA was extracted from the organic solution TE Buffer (400 μL) was added to the organic phase, vortexed, and centrifuged at 14,000 rpm for 3 minutes. The aqueous layer was collected, and two more extraction cycles were performed to maximize DNA recovery. The amount of DNA was quantified using a fluorometer and the fluorescent dye Hoechst 33258. To determine the in vitro release kinetics of DNA, scaffolds were placed in 500 μL of 1× phosphate-buffered saline (pH 7.4), and the solution was replaced at each time-point. DNA was again quantified using a fluorometer. The conformation of the DNA released from the scaffolds was analyzed with agarose gel electrophoresis. A digital image of the gel was taken and NIH image software was used to evaluate the fraction of DNA remaining in the supercoiled conformation.

### Evaluation of In Vivo Gene Expression

Scaffolds loaded with luciferase-encoding plasmid were sterilized in 70% ethanol, washed in 1640-RPMI islet medium, and then implanted into the epididymal fat pad of male C57BL/6 mice At desired time-points, scaffolds were retrieved and frozen over dry ice. The frozen tissue samples were cut up with small scissors, and 200 μL of cell culture lysis reagent (Promega) was added. Samples were placed on a rotator for 30 minutes. Samples were then snap frozen in liquid nitrogen, thawed in a 37 C water bath, and centrifuged at 14,000 rpm for 10 minutes at 4 C. The supernatant was removed and measured using luciferase assay reagent (Promega) and a luminometer with a 10 second integration time.

### Fabrication of Exendin-4 Loaded Scaffolds

Exendin-4 was encapsulated inside PLG microspheres using a double emulsion technique (w/o/w), and the drug-loaded microspheres were used to fabricate scaffolds as described above. Two PLG formulations were used to make microspheres that provide different release kinetics. The first formulation was 75% D,L latide/25% glycolide (i.v.=0.76 dl/g). The second formulation was an equal weight blend of the first formulation with 50% D,L lactide/50% glycolide (i.v.=0.45 dl/g). The PLG formulations were dissolved in dichloromethane to make 3% (w/w) solutions. An aqueous protein solution (17 μL total volume) containing 73 μg of exendin-4, 700 μg of bovine serum albumin (BSA), 50 mg/mL sucrose, and 3% wt MgCO3/wt. BSA was also prepared. The first emulsion was created by adding 500 μL of the PLG solution to the protein solution, and sonicating for 15 seconds at 40 W. The second emulsion was formed by pouring the first emulsion into 25 mL. of 5% PVA (with 50 mg/mL sucrose) and homogenizing for 45 seconds. The second emulsion was then poured into 15 mL of 1% PVA (with 50 mg/mL sucrose) and stirred for 1.5 hours to allow evaporation of dichloromethane. Microspheres were washed with deionized water, centrifuged at 4000 rpm for 10 min, and then frozen in liquid nitrogen and lyophilized overnight. The microsheres made with the first polymer formulation were used to construct the outer scaffold layers, while microspheres made with the second polymer formulation were used to construct the center layer of the scaffold.

### Characterization of Exendin-4 Release

Radiolabled (1-125) exendin-4 was used to measure protein release from scaffolds. Exendin-4 loaded microspheres were fabricated as described above, with radiolabeled exendin-4 added as a tracer. The radioactive microspheres were then used to fabricate scaffolds. To determine the in vitro release kinetics, scaffolds were placed in 1 mL of 1×PBS and incubated in a 37 C water bath. At desired time-points scaffolds were transferred to fresh PBS, and the activity of release buffer was determined using a gamma counter.

### Islet Transplantation on Exendin-4 Loaded Scaffolds

Exendin-4 loaded scaffolds were evaluated for their ability to improve the function/survival of transplanted islets in a synegenic mouse model. Male C57BL/6 mice were made diabetic with an intraperitoneal injection of 220 mg/kg of streptozotocin. Mice with blood glucose measurements>300 mg/dl on consecutive days were considered Diabetic. Islets were isolated from healthy C57BL/6 male mice as previously described (Blomeier, H. et al. Polymer scaffolds as synthetic microenvironments for extrahepatic islet transplantation. Transplantation 82, 452-459 (2006); Kaufman, D. B. et al. Effect of 15-deoxyspergualin on immediate function and long-term survival of transplanted islets in murine recipients of a marginal islet mass. Diabetes 43, 778-783 (1994); Hyon, S.H., Tracey, K. J. & Kaufman, D. B. Specific inhibition of macrophage-derived proinflammatory cytokine synthesis with a tetravalent guanylhydrazone CNI-1493 accelerates early islet graft function posttransplant. Transplant Proc 30, 409-410 (1998)). Scaffolds were sterilized in 70% ethanol and washed in 1640 RPMI islet growth medium. Islets were then seeded onto the scaffolds in a minimal volume of medium. Islet-loaded scaffolds were them implanted into the epididymal fat pad of diabetic recipient mice. Non-fasting blood glucose levels were monitored over time to evaluate the function of the transplanted islets

All publications and patents mentioned in the above specification are herein incorporated by reference in their entireties. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A scaffold composition for time-release delivery of biological or chemical agents to a subject, comprising: a) a substantially non-porous inner layer having a biological or chemical agent associated therewith; and b) porous outer layers having sufficient porosity to permit cellular ingrowth therein.

2. The composition of claim 1, wherein said substantially non-porous inner layer comprises said biological or chemical agent in encapsulated particles.

3. The composition of claim 2, wherein said encapsulated particles are microspheres.

4. The composition of claim 3, wherein said microspheres are poly(lactide-co-glycolide) microspheres.

5. The composition of claim 1, wherein said biological or chemical agent is a protein.

6. The composition of claim 1, wherein said biological or chemical agent is a cell.

7. The composition of claim 1, wherein said biological or chemical agents comprise exendin-4 and extracellular matrix proteins.

8. The composition of claim 7, further comprising pancreatic islet cells.

9. The composition of claim 1, wherein said inner layer is non-porous.

10. The composition of claim 1, wherein said inner layer is substantially free of salt.

11. The composition of claim 1, wherein said biological or chemical agent is nucleic acid.

12. The composition of claim 1, wherein said inner layer is composed of two or more different polymers.

13. The composition of claim 1, wherein said inner layer comprises two or more different biological or chemical agents.

14. The composition of claim 13, wherein each of said two or more different biological or chemical agents is contained in different microspheres, having different release rates.

15. The composition of claim 1, wherein said inner and outer layers are configured to permit slow-release of said biological or chemical agent over a period of at least 30 days.

16. The composition of claim 1, wherein said inner and outer layers are configured to permit slow-release of said biological or chemical agent over a period of at least 70 days.

17. A method of treating a subject, comprising: administering the composition of claim 1 to a subject.

18. The method of claim 17, wherein subject has type 1 diabetes.

19. The method of claim 17, wherein the composition increases blood glucose control and/or restores euglycemia.
